(19) United States
(12) Patent Application Publication
(10) Pub. No.: US 2010/0041027 A1
(43) Pub. Date: Feb. 18, 2010

(54) METHODS OF ASSESSING CORONARY ARTERY DISEASE

(75) Inventors: Mary Susan Burnett, Fairfax, VA (US); Joseph M. Devaney, Silver Spring, MD (US); Stephen E. Epstein, Rockville, MD (US)

Correspondence Address: WOLF GREENFIELD & SACKS, P.C., 600 ATLANTIC AVENUE, BOSTON, MA, 02210-2206, US

(73) Assignee: Medstar Research Institute, Inc., Hyattsville, MD (US)

(21) Appl. No.: 11/989,648

(22) PCT Filed: Jul. 31, 2006
(86) PCT No.: PCT/US2006/029920

§ 371 Date: Nov. 6, 2009

Related U.S. Application Data
(60) Provisional application No. 60/704,167, filed on Jul. 29, 2005.
(60) Provisional application No. 60/704,145, filed on Jul. 29, 2005.

(51) Int. Cl. C12Q 1/68

(52) U.S. Cl. 435/6

(57) ABSTRACT

An association between the Ala allele of the P12A variant of the human PPARγ gene and development of CAD, particularly premature CAD, in individuals, and specifically in women, particularly Caucasian women, is described, as are methods of assessing or predicting the likelihood or risk that an individual, such as a woman, will develop premature CAD. Single nucleotide polymorphisms in the human resistin gene, human resistin gene variants, gender-related increase in premature coronary artery disease, methods of assessing or aiding in assessing the risk that an individual will develop premature CAD, and methods of predicting the likelihood or aiding in predicting the likelihood that an individual will develop premature CAD are described.

### RELATED APPLICATIONS

This application claims the benefit of the filing date of U.S. Provisional application 60/704,145, entitled PPARγ2 P12A Polymorphism Association with Coronary Artery Disease, filed Jul. 29, 2005 and U.S. Provisional application 60/704,167, entitled Assessment of Gender-Related Increase in Premature Coronary Artery Disease, filed Jul. 29, 2005. The entire teachings and contents of both of these referenced provisional applications are incorporated herein by reference.

### GOVERNMENT FUNDING

The Strong Heart portion of this study was supported by cooperative agreement grants (U01-HL-41642, U01-HL-41652, and UL01-HL-41654) from the National Heart Lung, and Blood Institute. The government has certain rights in the invention.

### BACKGROUND OF THE INVENTION

Coronary artery disease is a multifactor disease that results in the deposition of atheromatous plaque and progressive luminal narrowing of the arteries that supply the heart muscle. Coronary artery disease (CAD) is a serious concern and it is difficult to predict who is at risk of developing atherosclerotic vascular disease, such as premature coronary artery disease. CAD is a serious concern in women and it is difficult to predict who is at risk of developing the condition Additional approaches for doing so are needed.

### SUMMARY OF THE INVENTION

The invention broadly relates to the diagnosis of vascular disease and further to methods of assessing or aiding in assessing the risk that an individual will develop CAD, such as premature CAD and methods of predicting or aiding in predicting that an individual will develop CAD, such as premature CAD. In specific embodiments, the invention relates to methods of assessing or aiding in assessing the risk that a woman will develop CAD, such as premature CAD, and methods of predicting or aiding in predicting that a woman will develop CAD, such as premature CAD. In further embodiments, the invention relates to methods of assessing or aiding in assessing the risk that a woman, such as a Caucasian woman, will develop CAD, such as premature CAD and methods of predicting or aiding in predicting that a woman, such as a Caucasian woman, will develop premature CAD.

In additional embodiments, the invention relates to methods of assessing or aiding in assessing the risk that a man will develop CAD, such as premature CAD, and methods of predicting or aiding in predicting that a man will develop CAD, such as premature CAD.

As described herein, Applicants have assessed variants in the PPARγ gene and their association with the development of CAD. Results of their assessments indicate that a PPARγ2 variant, PPARγ2 P12A, is useful as a marker for premature CAD in women. In addition, Applicants have shown that an allele of the P12A variant is associated with the development of CAD in women, specifically with the development of CAD in Caucasian women. The association between the variant and developing CAD before the age of 45 years (premature CAD) is particularly strong. The work described herein provides the basis for an additional approach to assessing the likelihood or risk of an increase in CAD, such as premature CAD, in individuals and particularly for doing so in women, especially Caucasian women.

As also described herein, Applicants have identified four novel single nucleotide polymorphisms (SNPs) in the human resistin gene. These four SNPs are designated T167C, G233A, C980G, and G1325A. Applicants have carried out assessments of the association of these SNPs with the CAD status of individuals. Results of their work indicate that at least one of the resistin variants (SNP C980G) is useful as a marker for premature CAD in men; C980G, is associated with an increased risk of premature CAD in men. The findings described herein not only further support the role of resistin in the development of CAD, but also provide the basis for an additional approach to assessing the likelihood or risk of an increase in premature CAD. In a specific embodiment, it provides the basis for assessing the likelihood or risk that a man will develop premature CAD.

### DETAILED DESCRIPTION OF THE INVENTION

This invention provides methods for diagnosing or aiding in diagnosing CAD and compositions useful for diagnosing or aiding in diagnosing CAD. In specific embodiments, the invention relates to methods of assessing or aiding in assessing the risk or likelihood that an individual will develop CAD, such as premature CAD. In further specific embodiments, the invention relates to methods of predicting or aiding in predicting that an individual will develop CAD, such as premature CAD. For example, the invention relates to methods of assessing or aiding in assessing the risk or likelihood that a woman will develop CAD, such as premature CAD, by determining the woman's status as to an allele of PPARγ2 associated with development of premature CAD, particularly determining whether the woman's DNA comprises the Ala allele of the PPARγ2 P12 variant. The invention also relates to methods of assessing or aiding in assessing the risk or likelihood that a woman will develop CAD, such as premature CAD, by determining whether the woman's DNA comprises the Ala allele of the PPARγ2 P12 variant. Both of these embodiments of the methods are particularly useful in Caucasian women.

As described in Example 2, among women, Applicants also tested for a relationship between MI and PPARγ genotype. Results showed that, adjusting for age, BMI and diabetes, the odds of MI increased 3.1 times with each Ala allele. The present invention also provides methods of predicting or aiding in predicting that an individual will experience a MI. For example, the invention relates to methods of assessing or aiding in assessing the risk or likelihood that a woman will experience a MI, by determining the woman's status as to an allele of PPARγ2 associated with increased occurrence of MI and, particularly, determining whether the woman's DNA comprises the Ala allele of the PPARγ2 P12 variant. The presence of the Ala allele of the P12 variant or the number of Ala alleles can be determined, using known methods. The invention also relates to methods of assessing or aiding in assessing the risk or likelihood that a woman will experience a MI, by determining whether the woman's DNA comprises the Ala allele of the PPARγ2 P12 variant or determining the number of Ala alleles in the woman's DNA.

In further specific embodiments, the invention relates to methods of predicting or aiding in predicting that a man will develop CAD, such as premature CAD. For example, the invention relates to methods of predicting or aiding in predicting that a man will develop CAD, such as premature CAD, by determining the man's status as to at least one resistin variant associated with development of premature CAD, particularly by determining whether the man's DNA comprises one or more copies of the G allele of resistin, such as by assessing the man's DNA for the presence of one or more copies of the C980G SNP, as described herein. The invention also relates to methods of assessing or aiding in assessing the risk or likelihood that a man will develop CAD, such as premature CAD, by determining the man's status as to at least one resistin variant associated with development of premature, particularly by determining whether the man's DNA comprises one or more copies of the G allele of resistin, such as by assessing the man's DNA for the presence of one or more copies of the C980G SNP, as described herein.

### Peroxisome Proliferator-Activated Receptor-Gamma (PPARγ)

The important influence of metabolic syndrome on atherogenesis has been one of the reasons for the enormous investigational and clinical attention focused on the peroxisome proliferator-activated receptors (PPARs), which appear to be major players in several of the components of the metabolic syndrome. The PPARs are ligand activated nuclear transcription factors. To date, three isoforms have been identified: PPARα, PPARγ, and PPARδ. These subtypes have both separate and overlapping expression patterns and biological activities. The work described herein focuses on whether genetic polymorphisms of one these three isoforms—PPARγ—are present that are functionally relevant, can serve as a potent biomarker of atherosclerotic risk, and is pharmacologically targetable.

Peroxisome proliferator-activated receptor-gamma (PPARγ) belongs to the nuclear hormone receptor superfamily of PPARs and is a central regulator of adipose cell differentiation. PPARγ2 is a crucial molecule in atherogenesis because it is associated with metabolic risk factors such as obesity and diabetes. Applicants hypothesized that variants in the PPARγ gene are associated with the development of CAD and investigated the role of PPARγ2 gene polymorphisms in coronary artery disease (CAD).

Applicants have shown that the Ala allele of the P12A variant is associated with development of CAD in Caucasian women and that this association is particularly strong for women, especially Caucasian women, developing CAD <45 years of age.

In this embodiment, the present invention relates to methods of assessing the risk or predicting the likelihood that an individual will develop CAD, particularly premature CAD (CAD prior to approximately 45 years of age). In the methods, the occurrence of the Ala allele of the P12A variant is assessed in DNA obtained from an individual, particularly a Caucasian woman. The presence of at least one copy of the Ala allele of the P12A variant in the DNA indicates that the individual (e.g. a woman, such as a Caucasian woman) from whom it was obtained is at increased risk of developing CAD, such as premature CAD. If an individual is determined to be at increased risk of developing premature CAD, other risk factors, such as diet, weight, smoking habits, and exercise level, can be addressed in order to contribute to reducing the risk of developing premature CAD or at least to delay the onset of CAD.

One embodiment of the invention is a method of assessing or aiding in assessing the risk that an individual (e.g., a woman, such as a Caucasian woman) will develop CAD, particularly premature CAD. The method comprises obtaining a biological sample (e.g., blood, serum or plasma) from an individual (e.g., a woman, such as a Caucasian woman) to be assessed for the risk of developing premature CAD and analyzing the sample (e.g., DNA in the sample) for the presence of one or more copies (at least one copy) of the Ala allele of the human PPARγ gene, wherein if the sample contains at least one (one or more; one or two) copies of the Ala allele, the individual is at increased risk for developing CAD, such as premature CAD. In a further embodiment, the invention is a method of predicting or aiding in predicting the likelihood that an individual will develop CAD, such as premature CAD, comprising obtaining a biological sample (e.g., blood, serum or plasma) from an individual (e.g., a woman, such as a Caucasian woman) for whom the risk of developing CAD, particularly premature CAD, is to be predicted (assessed) and determining if one or two copies (at least one copy) of the Ala allele of the human PPARγ gene is/are present in the sample (in DNA in the sample), wherein if the sample contains at least one (one or more; one or two) copies of the Ala allele, the likelihood that the individual will develop CAD, particularly premature CAD, is greater than if the sample does not contain the Ala allele. In the methods of the present invention, the samples can be processed in any manner which renders DNA available for analysis or assessment and DNA can be detected by known methods, such as hybridization methods or amplification-based (e.g., PCR) methods. Individuals assessed by the present methods can be females of any age, particularly any age younger than about 45 years; children, youth and adults can be assessed. The terms woman and female are used interchangeably.

The present invention relates to methods of assessing or aiding in assessing the risk and methods of predicting or aiding in predicting the likelihood that a woman, such as a Caucasian woman, will develop CAD, particularly premature CAD (CAD prior to approximately 45 years of age). In the methods, the occurrence of the Ala allele of the P12A variant is assessed in DNA obtained from a woman, such as a Caucasian woman. The presence of at least one copy of the Ala allele of the P12A variant in the DNA indicates that the woman from whom it was obtained is at increased risk of developing CAD, such as premature CAD. If a woman is determined to be at increased risk of developing CAD, such as premature CAD, other risk factors, such as diet, weight, smoking habits, and exercise level, can be addressed in order to contribute to reducing the risk of developing CAD, such as premature CAD or at least to delay the onset of CAD, such as premature CAD.

One embodiment of the invention is a method of assessing or aiding in assessing the risk that a woman will develop premature CAD. The method comprises obtaining a biological sample (e.g., blood, serum or plasma)) from a woman to be assessed for the risk of developing premature CAD and analyzing the sample (DNA in the sample) for the presence of one or more copies (at least one copy) of the Ala allele of the human PPARγ gene, wherein if the sample contains at least one (one or more; one or two) copies of the Ala allele, the woman is at increased risk for developing CAD, such as premature CAD. In a further embodiment, the invention is a method of predicting or aiding in predicting the likelihood that a woman will develop CAD, such as premature CAD, comprising obtaining a biological sample (e.g., blood, serum or plasma) from a woman for whom the risk of developing CAD, such as premature CAD is to be predicted (assessed) and determining if one or two copies (at least one copy) of the Ala allele of the human PPARγ gene is/are present in the sample (DNA in the sample), wherein if the sample contains at least one (one or more; one or two) copies of the Ala allele, the likelihood that the woman will develop CAD, such as premature CAD, is greater than if the sample does not contain the Ala allele. In the methods of the present invention, the samples can be processed in any manner which renders DNA available for analysis or assessment and DNA can be detected by known methods, such as hybridization methods or amplification-based (e.g., PCR) methods. Women assessed by the present methods can be of any age, particularly any age younger than about 45 years; children, youth and adults can be assessed. In specific embodiments, the methods of the present invention are methods of assessing the risk or aiding in assessing the risk of premature CAD in women and, more specifically in Caucasian women, and methods of predicting or aiding in predicting or aiding in predicting the likelihood that a woman, and more specifically a Caucasian woman, will develop premature CAD.

PPAR-γ is a transcription factor in the nuclear receptor family, and can be found in three isoforms, PPARγ1, PPAR-γ2, and PPAR-γ3. While the tissue distribution of these isoforms varies, all three forms are found in adipose tissue, in which PPAR-γ2 is the most prevalent. PPARs are importantly involved in regulating adipocyte differentiation and fatty acid and lipid metabolism. Furthermore, they are involved in glucose homeostasis, and are targets for the thiazolidinedione drugs.

As a transcriptional activator, PPAR-γ is able to induce the transcription of various target genes involved in adipocyte biology, as well as glucose homeostasis. It is, therefore, reasonable to suggest that polymorphisms within the PPAR-γ gene could lead to alterations in not only PPAR-γ activity, but also in the activity of various downstream genes that are targets of PPAR-γ activation. Previous publications suggest that this P12A variant is associated with reduced PPARγ2 activity (Deeb, et al), as well as a lower lipoprotein lipase (LPL) activity in vivo (Schneider et al). LPL contributes to lipid metabolism by hydrolyzing triglycerides in circulating lipoproteins. Low LPL activity has been linked to both CAD (Benlian et. al., Clee et. al, Wittrup et. al.) and endothelial dysfunction (Kastelein et. al.). These findings support the hypothesis that decreased PPAR-γ activity resulting from the P12A polymorphism can have downstream effects, such as decreased LPL activity, which has been shown to increase the risk of CAD.

PPARs are molecular targets for the thiazolidinediones (TZDs), which are currently used to treat type 2 diabetes in individuals whose diabetes cannot be controlled by diet and exercise alone. The TZDs act by increasing the body's sensitivity to insulin, in part by increasing PPAR-γ activity. It should therefore be possible to decrease CAD risk in patients with the P12A polymorphism by treating with a TZD, or other PPARγ agonist, which would effectively increase PPARγ activity. Specifically, individuals with the P12A variant are likely to have decreased PPAR-γ activity, which would lead to numerous changes in the expression of downstream target genes, such as decreases in LPL activity. Since it is known that decreased LPL activity is associated with atherosclerosis, a therapy increasing LPL activity should improve atherosclerotic disease. The present invention further relates to a method of decreasing CAD risk in individuals, particularly women (e.g., Caucasian women), with the P12A polymorphism by administering to the individual a therapeutically effective amount of a PPARγ agonist, such as TZD. A therapeutically effective amount is one which increases PPARγ activity sufficiently to, for example, correct the effect(s) on cells caused by a PPARγ deficiency (e.g., decreased LPL activity).

The following references are those cited in the above section entitled Peroxisome proliferator-activated receptor-gamma (PPARγ).

### REFERENCES

Schneider J, Kreuzer J, Hamann A, Nawroth P P, Dugi K A. The proline 12 alanine substitution in the peroxisome proliferator-activated receptor-γ2 gene is associated with lower lipoprotein lipase activity in vivo. Diabetes 2002; 51:867-870.
Benlian P, De Gennes J L, Foubert L, Zhang H, Gagne S E, Hayden M. Premature atherosclerosis in patients with familial chylomicronemia caused by mutations in the lipoprotein lipase gene. N Engl J Med 1996; 335:848-854.
Clee S M, Bissada N, Miao F, Miao L, Marais A D, Henderson H E, Steures P, McManus J, McManus B, LeBoeuf R C, Kastelein J J, Hayden M R. Plasma and vessel wall lipoprotein lipase have different roles in atherosclerosis. J Lipid Res 2000; 41:521-531.
Wittrup H H, Tybjaerg-Hansen A, Steffensen R, Deeb S S, Brunzell J D, Jensen G, Nordestgaard B G. Mutations in the lipoprotein lipase gene associated with ischemic heart disease in men: the Copenhagen City Heart study. Arterioscler Thromb Vasc Biol 1999; 19:1535-1540.
Kastelein J J, Jukema J W, Zwinderman A H, Clee S, van Boven A J, Jansen H, Rabelink T J, Peters R J, Lie K I, Liu G, Bruschke A V, Hayden M R. Lipoprotein lipase activity is associated with severity of angina pectoris. Circulation 2000; 102:1629-1633.
Al-Shali K Z, House A A, Hanley A J G, Khan H M R, Harris S B, Zinman B, Mamakeesick M, Fenster A, Spence J D, Hegele R A. Genetic variation in PPARG encoding peroxisome proliferators-activated receptor γ associated with carotid atherosclerosis. Stroke 2004; 35:2036-2040.
Temelkova-Kurktsheiev T, Hanefeld M, Chinetti G, Zawadzki C, Haulon S, Kubaszek A, Koehler C, Leonhardt W, Staels B, Laakso M. Ala12Ala genotype of the peroxisome proliferators-activated receptor γ2 protects against atherosclerosis. J Clin Endocrinol Metab 2004; 89:4238-4242.

### Human Resistin Gene Single Nucleotide Polymorphisms

Applicants have identified four novel single nucleotide polymorphisms (SNPs) in the human resistin gene. These four SNPs are designated T167C, G233A, C980G, and G1325A. Assessments of the association of these SNPs with the CAD status of individuals indicate that at least one of the resistin variants (SNP C980G) is useful as a marker for premature CAD in men; C980G, is associated with an increased risk of premature CAD in men. The findings described herein not only further support the role of resistin in the development of CAD, but also provide the basis for an additional approach to assessing the likelihood or risk of an increase in premature CAD. In a specific embodiment, it provides the basis for assessing the likelihood or risk that a man will develop premature CAD.

The novel C980G SNP in resistin is associated with an increased risk of premature CAD in men, a finding that further implicates a role of resistin in the development of CAD. Ongoing assessment of a separate population supports the use of this resistin variant as a more general marker (one useful in assessing or aiding in assessing or predicting or aiding in predicting the likelihood that an individual (male or female) will develop premature CAD).

The present invention relates to methods of assessing the risk or predicting the likelihood that an individual will develop CAD, particularly premature CAD (CAD prior to approximately 45 years of age). In the methods, the occurrence of one or two copies of the G allele is assessed in DNA obtained from an individual. The presence of one or two copies of the G allele in the DNA indicates that the individual from whom it was obtained is at increased risk of developing premature CAD. If an individual is determined to be at increased risk of developing premature CAD, other risk factors, such as diet, weight, smoking habits, and exercise level, can be addressed in order to contribute to reducing the risk of developing premature CAD or at least to delay the onset of CAD. In specific embodiments, the present invention relates to methods of assessing the risk or predicting the likelihood that a man will develop CAD, particularly premature CAD (CAD prior to approximately 45 years of age). In the methods, the occurrence of one or two copies of the G allele is assessed in DNA obtained from a man. The presence of one or two copies of the G allele in the DNA indicates that the man from whom it was obtained is at increased risk of developing premature CAD.

One embodiment of the invention is a method of assessing or aiding in assessing the risk that an individual (particularly a male) will develop premature CAD. The method comprises obtaining a biological sample (e.g., blood, serum or plasma)) from an individual (a man) to be assessed for the risk of developing premature CAD and analyzing the sample (e.g., DNA in the sample) for the presence of one or two copies (at least one copy) of the G allele of the human resistin gene, wherein if the sample contains at least one (one or more; one or two) copies of the G allele, the individual (e.g., the man) is at increased risk for developing premature CAD. In a further embodiment, the invention is a method of predicting the likelihood that an individual (a man) will develop premature CAD, comprising obtaining a biological sample (e.g., blood, serum or plasma) from an individual (a man) for whom the risk of developing premature CAD is to be predicted (assessed) and determining if one or two copies (at least one copy) of the G allele of the human resistin gene is/are present in the sample (DNA in the sample), wherein if the sample contains at least one (one or more; one or two) copies of the G allele, the likelihood that the individual will develop premature CAD is greater than if the sample does not contain the G allele. In the methods of the present invention, the samples can be processed in any manner which renders DNA available for analysis or assessment and DNA can be detected by known methods, such as hybridization methods or amplification-based (e.g., PCR) methods. Individuals assessed by the present methods can be of any age, particularly any age younger than about 45 years; children, youth and adults can be assessed.

One specific embodiment of the invention is a method of assessing or aiding in assessing the risk that a man will develop premature CAD. The method comprises obtaining a biological sample (e.g., blood, serum or plasma)) from a man to be assessed for the risk of developing premature CAD and analyzing the sample (DNA in the sample) for the presence of one or two copies (at least one copy) of the G allele of the human resistin gene, wherein if the sample contains at least one (one or more; one or two) copies of the G allele, the man is at increased risk for developing premature CAD. In a further embodiment, the invention is a method of predicting the likelihood that a man will develop premature CAD, comprising obtaining a biological sample (e.g., blood, serum or plasma) from a man for whom the risk of developing premature CAD is to be predicted (assessed) and determining if one or two copies (at least one copy) of the G allele of the human resistin gene is/are present in the sample (DNA in the sample), wherein if the sample contains at least one (one or more; one or two) copies of the G allele, the likelihood that the man will develop premature CAD is greater than if the sample does not contain the G allele. In the methods of the present invention, the samples can be processed in any manner which renders DNA available for analysis or assessment and DNA can be detected by known methods, such as hybridization methods or amplification-based (e.g., PCR) methods. As used herein, the term “man” includes males of any age and particularly those younger than about 45 years. Individuals assessed by the present methods can be males of any age, particularly any age younger than about 45 years; children, youth and adults can be assessed. The terms man/men and male/males are used interchangeably.

### EXEMPLIFICATION

### Example 1

### Preliminary Assessment of Two PPARγ Variants

Two PPARγ variants (Pro12Ala and C2821T) were genotyped in a population of 688 individuals who underwent coronary angiography at the Washington Hospital Center. Premature CAD patients were defined as individuals diagnosed with CAD prior to age 45. Individuals under 45 years with no angiographic evidence of CAD served as controls.

Of the 688 individuals in this study, 485 (70%) had CAD; 256 of these (37%) had premature CAD (<age 45). The frequency of allele Ala in African Americans was 0.010 compared to 0.125 in Caucasians (p<0.0001). Hardy-Weinberg equilibrium was not violated in either the Caucasian or African American subgroups (χ2 p=0.64, p=0.90, respectively). Adjusting for race, estimated age, and BMI category (<25, 25-30, 30<), there was no association between presence of an Ala allele and either early or all category CAD. Stratifying by race and gender, Caucasian and African-American men still showed no association, while in African American women (n=70) there were no copies of the Ala allele. However, among Caucasian women (n=164) odds of CAD at any age increased significantly with each copy of the Ala allele inherited (OR=3.0, 95% CI 1.3-7.2, p=0.011). Adjusted odds of premature CAD in Caucasian women with a Ala allele were even higher compared to those with the Pro/Pro genotype (OR=5.9, 95% CI 1.8-18.5, p=0.002).

The PPARγ-C2821T variant was also successfully genotyped in 664 people with data on CAD; however, no associations were found with CAD.

### Example 2

### Further Assessment of Two PPARγ Variants

This example presents results of assessment of an enlarged population described in Example 1. Two PPARγ variants (Pro12Ala and C2821T) were genotyped in a population of 697 individuals admitted for cardiac catheterization at the Washington Hospital Center. After stratifying by race and gender, among Caucasian women (n=164) odds of CAD at any age increased significantly with each copy of the Ala allele inherited in the Pro12Ala position (OR=5.0, 95% CI 1.6-15.5, p=0.005). Adjusted odds of premature CAD (presentation <45 yrs) in Caucasian women with an Ala allele were even higher compared to those with the Pro12Pro genotype (OR=11.0, 95% CI 2.0-58.6, p=0.005). In addition, the Ala alelle was associated with mycardial infarction (MI) in women (OR=2.3, 95% CI 1.2-4.5, p=0.02). For confirmation, a second population (American Indians; n=3,871) enrolled in the Strong Heart Study was genotyped. American Indian women carrying the Ala allele showed a higher incidence of MI (OR=1.53, 95% CI 1.07-2.19 P=0.02).

No associations were found with the -C2821T variant and CAD or MI. Similar associations were not seen in men.

Conclusions: Caucasian women with the Ala allele of the Pro12Ala variant in PPARγ2 have a high risk of developing CAD, and both Caucasian and American Indian women are at increased risk of developing MI.

### Methods

### Washington Hospital Center Cohort

Under a protocol approved by the Institutional Review Board at the Washington Hospital Center, blood was collected from consenting individuals undergoing coronary angiography at the Washington Hospital Center. Individuals were enrolled between January 2001 and December 2003 (n=697). CAD patients (n=471) were defined as individuals diagnosed with CAD by angiography (>50% stenosis in at least one vessel, or had suffered a myocardial infarction). Patients with premature CAD were defined as individuals who were diagnosed with CAD by angiography or had suffered a myocardial infarction prior to the age of 45 (n=249). Individuals ≧45 years of age with no more than 20% stenosis in any major coronary vessel, as determined by angiography, served as controls (n=202). DNA was isolated from whole blood using PurGene Reagents (Gentra), and stored at −80° C. until genotyping.

### Strong Heart Study

Design, survey methods and laboratory techniques of the SHS have been reported previously⁽¹⁶⁾. In brief, the SHS is a population-based study of 4549 men and women of American Indian heritage from three geographic areas (Arizona, North and South Dakota, and Oklahoma)⁽¹⁶⁾. Participants were invited to a study examination on three occasions (SH1:1988-92; SH2:1993-1995; SH3:1997-1999) and remain under continued surveillance (for the purposes of this report to the end of 2000) for development of vascular disease.

The process used to ascertain fatal and non-fatal coronary heart disease (CHD) events has been described previously^{(17,16)}. For the purpose of analyses presented herein, participants with cardiovascular disease (either CHD or stroke at baseline) were excluded, to allow analysis of incident CHD and myocardial infarction.

### Genotyping

Genotyping was performed using a TaqMan allele discrimination assay that employs the 5′ nuclease activity of Taq polymerase to detect a fluorescent reporter signal generated during PCR reactions. Both alleles were detected simultaneously using allele-specific oligonucleotides labeled with different fluorophores, and genotypes determined by the ratio of the two fluorophores used. The allele-specific PCR reactions for the PPARγ2 Pro12Ala variant contained 5 ng DNA, 900 nM polymerase chain reaction (PCR) primers (F: GTTATGGGTGAAACTCTGGGAGATT and R: GCAGACAGTGTATCAGTGAAGGAAT), 200 nM fluorescent allele discrimination probes (Pro allele: VIC-CTCCTATTGACCCAGAAAG; Ala allele: FAM-CTATTGACGCAGAAAG) and TaqMan® Universal PCR Master Mix, No AmpErase® UNG (Applied Biosystems, Foster City, Calif., USA) in a final volume of 10 μl. The PCR was performed over 10 min at 95° C. (activation of polymerase), and 44 cycles of 15 s at 92° C. (denaturation) and 1 min at an annealing temperature of 60° C. Reactions were set up using a MWG robot, and fluorescence ratios and allele calling done using an ABI 7900. To test for reproducibility of the genotyping assay, 96 DNA samples were tested three times independently, and genotypes compared. There was 100% concordance of genotypes in all three replicates, with no dropouts, no calls, or ambiguous calls,

### Statistical Analysis

The presence of Hardy-Weinberg equilibrium was tested separately in white non-Hispanics and African Americans using the chi-square method.

### Washington Hospital Center

Analyses in the initial data set were stratified by ethnicity. Data from white non-Hispanics and African American participants were analyzed, but Asians and American Indians (n=9 and 12, respectively), were excluded from analysis because of small numbers.

Birthdates were provided, but exact exam dates were not available to calculate precise age at examination. Since patients were examined between January 2001 and December 2003, an exam date of Jan. 1, 2002 was used to estimate age at exam. Information on participant's birth date, race, gender, height, weight and diabetic state at the time of exam was collected. Body mass index (BMI) was calculated as weight/height² (kg/m²). Differences between unadjusted baseline clinical characteristics of the WHC cohort were compared using t-tests, with two-tailed p-values considered significant at the 0.05 level. The odds of CAD and MI associated with the dominant model of Ala alleles inherited were calculated using logistic regression. Several logistic regression models were run with different covariates including age, BMI, and presence of diabetes. Each analysis was stratified by gender and reported for Caucasians only due to small sample sizes in other ethnic groups.

### Strong Heart Study

DNA samples were available for 3747 participants (82%). Differences between unadjusted baseline clinical characteristics of the SHS cohort were compared using t-tests, with two-tailed p-values considered significant at the 0.05 level. The odds of CAD and MI associated with the dominant model of Ala alleles inherited were calculated using logistic regression. Several logistic regression models were run with different covariates including age, BMI, study center, and fasting glucose level. Each analysis was stratified by gender and all participants were of a single ethnic group, American-Indian.

All analyses used SAS (SAS institute, Cary, N.C., USA) and Stata (StataCorp, College Station, Tex., USA) software.

### Results

### Washington Hospital Center

Relationship Between Pro12Ala variant and CAD or Early Onset CAD

The Pro12Ala variant (CCG=proline; GCG=alanine) was successfully genotyped in 697 individuals (520 white non-Hispanics and 144 African Americans). 471 (70%) participants were diagnosed with CAD; 249 of these (37%) had premature CAD. There were no significant associations of genotype with age, BMI, diabetes, and serum HDL, LDL and triglyceride levels (Table 1). Among women, a history of diabetes and lower levels of HDL was significantly associated with prevalent CAD, while triglyceride levels were significantly higher in men with CAD than in men without CAD (data not shown).

The Ala allele (G) was much less frequent in African Americans (1%) than in Caucasians (12.5%, p<0.0001). Hardy-Weinberg equilibrium was not violated in either the Caucasian or African American subgroups (p=0.64, p=0.90, respectively). Across all participants, there was no observed association between the number of Ala alleles carried and CAD or early onset CAD (OR All CAD=1.4, 95% CI 0.9-2.7, p=0.14; OR Early CAD=1.5, 95% CI 0.8-2.7, p=0.23) (Table 2).

Stratifying by race and gender, adjusting for age, BMI and diabetes, Caucasian men and African-American men (data not shown) showed no association between Ala alleles and CAD. Among the African American women in the sample (n=70), there were no copies of the Ala allele. However, among Caucasian women (n=147) odds of CAD (adjusted for age, BMI, and diabetes) increased significantly with each copy of the Ala allele inherited (OR=4.1, 95% CI 1.5-11.3, p=0.007) (Table 3A). Including HDL or triglycerides did not improve the fit of the logistic regression or alter the findings substantially, so these covariates were not included in the final models.

There were no significant differences in Ala allele frequencies between participants over and under the age of 65, providing no evidence for survival differences across genotypes. The presence of one or more Ala alleles was not associated with diabetes in the cohort or any age- or race-specific subgroups. There was also no significant difference in BMI by genotype, adjusting for age and stratifying by race and gender.

Relationship Between MI and PPARγ Genotype

Among women, Applicants also tested for a relationship between MI and PPARγ genotype. Of 231 women with data on both PPARγ genotype and their history of myocardial infarction, 64 (28%) had a history of MI. Adjusting for age, BMI and diabetes, the odds of MI increased 3.1 times (95% CI 1.3-7.4, p=0.008) with each Ala allele (Table 3C).

### Strong Heart Study

Relationship Between Pro12Ala Variant and CHD or MI

In Strong Heart participants, there were no significant relationships between PPARγ genotype and sex, age, fasting glucose, treatment for hypertension, SBP, DBP, or total cholesterol (Table 2) in women, but a significant relationship between PPARγ and blood glucose (p=0.005) was seen. In men, only MBI was shown to be significantly related to PPARγ genotype (Table 2).

For all male participants there were no significant relationships of Pro12Ala genotype with either incident CHD or myocardial infarction (Tables 4B and 4D). For female participants, (n=1523) odds of MI (adjusted for age, BMI and fasting glucose) increased significantly with a copy of the Ala allele inherited (OR=1.8, 95% CI 1.1-3.1, p-0.030) (Table 4C). No significant association was seen between CAD and PPARγ genotype in Caucasian females (Table 4A).

These findings have mechanistic support from a number of in vitro and in vivo studies. Thus, PPARγ itself appears to have atheroprotective effects. Its activation reduces inflammatory cytokine production by monocytes/macrophages and T lymphocytes, and reduces adhesion molecule, MMP and chemokine production by endothelial cells (18, 19, 10, 12, 20, 21). Moreover, PPARγ agonists reduce atherosclerosis in various animal models, (7, 22, 23, 24) and decrease inflammation and vascular complications such as restenosis, in patients with diabetes (25, 26). The fact that PPARγ2 has atheroprotective effects can be mechanistically related to our findings that the Pro12Ala polymorphism is associated with CAD/MI. This polymorphism has been associated with a decrease in activity of PPARγ2(9, 27), as well as the downstream target lipoprotein lipase (LPL)(9, 28). LPL hydrolyzes triglycerides in circulating lipoproteins, and low levels of LPL activity have been linked to CAD. (29, 30, 31) Thus, Applicants postulate that the association between the Pro12Ala and CAD/MI is at least in part caused by the fact that the Ala12 allele results in reduced PPARγ2 activity and thereby its downstream targets, such as LPL.

While it is interesting that the findings are gender specific, it should not be surprising that polymorphisms may have different effects in men and women. Weiss et. al. have recently published a study in which sedentary men and women were genotyped for the Pro12Ala variant to determine if this polymorphism predicted: 1) insulin action, and 2) improvements in insulin sensitivity in response to endurance exercise training (32). The authors found that insulin-induced glucose disposal was lower in men with the Pro12Ala variant compared to those with Pro12Pro. However, men were particularly responsive to the effects of endurance training-induced improvements in insulin action. These effects were not seen in the women.

Others have reported different effects of single nucleotide polymorphisms (SNPs) between men and women in various contexts. For example, a SNP of the mu-opioid receptor gene (OPRM1) has been associated with differences in heat pain perception, such that under certain conditions, the rare allele is associated with lower pain ratings in men, but higher pain ratings in women.(33) Gender-specific associations between a SNP in GPR50, an X-linked orphan G protein-coupled receptor, and biopolar affective disorder in women has been recently described. (34) It was also reported in a large study of 718 individuals, that SNPs in the genes encoding estrogen receptors alpha and beta may be associated with genetic regulation of blood pressure in men, but not in women (35). Finally, an intriguing experimental finding that relates to gender specific effects of PPARγ on atherosclerosis is the observation that female mice exhibited less atherosclerosis reduction in response to PPARγ ligand treatment (36), suggesting that such factors as hormonal status might modulate the effects of this protein—or altered forms of the protein—on propensity for atherosclerosis.

The findings of the present investigation have important clinical implications. They suggest that in women, particularly Caucasian women, genotyping for the Pro12Ala would provide a potent biomarker for risk of developing premature CAD. Most importantly, because it has been shown that this allele conveys decreased biological activity for PPARγ, the findings strongly suggest that studies be initiated to determine whether treatment with PPARγ ligands in women with this allele effectively modulate either CAD development or the appearance of surrogate markers of CAD. If such studies confirm a beneficial effect, women with the Pro12Ala allele might be considered candidates for treatment using ligands of PPARγ.

In conclusion, Applicants report that the Pro12Ala polymorphism in the PPARγ2 gene is associated with an increased risk of CAD and MI in Caucasian women, and an increased risk of incident MI in American Indian women. The increased risk for premature CAD is particularly robust. While the exact mechanisms of action have yet to be elucidated, it is likely that multiple complex interactions between PPARγ2 and different downstream gene products are involved, as well as interactions with pathways not directly related to the PPARγ2 pathway. Since the Pro12Ala variant is associated with a decrease in PPARγ activity, it is PPARγ agonists, such as TZDs, may provide therapeutic benefits to women identified with this polymorphism.

### Example 3

### Assessment of the Relationship Between Premature CAD and Single Nucleotide Polymorphisms in the Human Resistin Gene

DNA was collected from individuals undergoing coronary angiography. Premature CAD patients were defined as those diagnosed with CAD prior to age 45. Individuals under 45 years of age with no angiographic evidence of CAD served as controls. Samples were genotyped using TaqMan assays. Hardy-Weinberg equilibrium (HWE) was tested using the chi-square method. Allele and genotype frequencies, HWE, and risk of CAD associated with number of minor alleles (the minor allele as a dominant factor and as a recessive factor of the SNP) were calculated for the entire dataset, and separately for Caucasians, African Americans, men, and women. Odds ratios of CAD associated with genotype were calculated using logistic regression, adjusting for age, BMI, and gender and race where appropriate.

A total of 624 people were genotyped. Analysis showed that 437 people (70%) had CAD; 224 of these (36%) had premature CAD. For the C980G SNP, G allele frequency in African Americans was 0.10 compared to 0.41 in Caucasians (p<0.00001). HWE was not violated in the Caucasian and African American subgroups (chi-squares; p=0.53, p=0.53 respectively). Adjusting for race, estimated age, and BMI category (<25, 25-30, 30<), the presence of 1 or 2 copies of the G allele (dominant model) was not significantly associated with increased risk of all case CAD. However, all case risk did tend to be increased in men (OR=1.7, 95% CI 0.99-2.7, p=0.51). Most interestingly, in men with 1 or two copies of the G allele the adjusted odds of premature CAD was significantly increased (OR=2.0, 95% Cl 1.1-3.7, p=0.03). The association was not observed in women. No associations with CAD were observed for the remaining three SNPs.

TABLE 1
Baseline Clinical Characteristics for Washington Hospital Center Cohort

| | Females | | | | | Males | | | | |
|---|---|---|---|---|---|---|---|---|---|---|
| | PPAR gamma (P12A) Genotype | | | | | PPAR gamma (P12A) Genotype | | | | |
| | | | | CG (Pro/ | | | | | CG (Pro/ | |
| | | | | Ala) | | | | | Ala) | |
| | | CC | | GG | | | CC | | GG | |
| Characteristic | N | (Pro/Pro) | N | (Ala/Ala) | P* | N | (Pro/Pro) | N | (Ala/Ala) | P* |
| Age (years) | 178 | 54.63 ± 12.77 | 34 | 58.58 ± 13.34 | 0.10 | 321 | 53.14 ± 12.54 | 76 | 53.32 ± 13.06 | 0.91 |
| BMI (kg/m²) | 178 | 30.91 ± 7.07 | 34 | 31.77 ± 8.49 | 0.53 | 321 | 30.08 ± 6.07 | 76 | 30.20 ± 6.68 | 0.88 |
| Blood glucose | 147 | 130.37 ± 57.08 | 29 | 136.69 ± 72.08 | 0.60 | 271 | 122.36 ± 48.50 | 62 | 113.64 ± 31.57 | 0.18 |
| (mg/dl) | | | | | | | | | | |
| HDL | 115 | 51.34 ± 15.91 | 28 | 52.18 ± 17.69 | 0.81 | 208 | 42.15 ± 12.18 | 56 | 42.75 ± 11.84 | 0.74 |
| LDL | 107 | 125.00 ± 122.54 | 24 | 100.12 ± 44.84 | 0.33 | 180 | 104.82 ± 34.36 | 52 | 96.10 ± 31.78 | 0.10 |
| Triglycerides | 116 | 145.95 ± 107.04 | 28 | 127.11 ± 51.77 | 0.37 | 211 | 161.28 ± 115.84 | 56 | 169 ± 103.69 | 0.65 |
| Cholesterol | 116 | 191.06 ± 52.87 | 29 | 172.62 ± 53.02 | 0.09 | 213 | 171.30 ± 45.57 | 58 | 174.10 ± 35.01 | 0.66 |
| Diabetes (%) | 178 | 83.9% | 34 | 16.1% | | 321 | 80.9% | 76 | 19.1% | |

*p-value from comparison of characteristic between PPAR gamma (P12A) genotypes

TABLE 2
Baseline Clinical Characteristics for Strong Heart Study Cohort

| | Females | | | | | Males | | | | |
|---|---|---|---|---|---|---|---|---|---|---|
| | PPAR gamma (P12A) Genotype | | | | | PPAR gamma (P12A) Genotype | | | | |
| | | | | CG (Pro/ | | | | | CG (Pro/ | |
| | | | | Ala) | | | | | Ala) | |
| | | CC | | GG | | | CC | | GG | |
| Characteristic | N | (Pro/Pro) | N | (Ala/Ala) | P* | N | (Pro/Pro) | N | (Ala/Ala) | P* |
| Age (years) | 1755 | 56.41 ± 7.96 | 516 | 56.73 ± 8.08 | 0.42 | 1146 | 55.52 ± 7.81 | 306 | 55.39 ± 8.12 | 0.80 |
| BMI (kg/m²) | 1751 | 31.49 ± 6.46 | 516 | 31.56 ± 6.51 | 0.81 | 1138 | 29.71 ± 5.98 | 305 | 30.79 ± 6.06 | 0.005 |
| Blood glucose | 1177 | 186.82 ± 110.03 | 346 | 168.49 ± 94.89 | 0.005 | 846 | 163.74 ± 104.02 | 212 | 150.08 ± 79.18 | 0.07 |
| (mg/dl) | | | | | | | | | | |
| HDL | 1507 | 47.39 ± 12.76 | 451 | 48.17 ± 13.22 | 0.26 | 907 | 43.20 ± 12.60 | 247 | 42.12 ± 14.49 | 0.25 |
| LDL | 1501 | 113.84 ± 31.89 | 451 | 113.53 ± 30.67 | 0.85 | 904 | 114.45 ± 32.36 | 246 | 115.54 ± 33.34 | 0.64 |
| Triglycerides | 1506 | 147.21 ± 103.43 | 451 | 141.22 ± 103.44 | 0.28 | 906 | 152.66 ± 218.51 | 248 | 144.29 ± 106.41 | 0.56 |
| Diabetes (%) | 752 | 51.6% | 201 | 46.7% | | 357 | 41.1% | 85 | 36.3% | |

*p-value from comparison of characteristic

TABLE 3A
Logistic regression analysis of PPAR gamma (P12A) recessive models with
PCAD/CAD in WHC Caucasian* females

| | PPAR γ | | | | | |
|---|---|---|---|---|---|---|
| | (P12A) | N | | PPAR γ (P12A) Factor | | |
| Covariates | Genotype | (controls) | N (CAD) | OR | p-value | 95% C.I. |
| Age | CC | 48 | 65 | 1.00** | | |
| | CG/GG | 6 | 28 | 2.82 | 0.017 | 1.201-6.636 |
| Age and BMI | CC | 48 | 65 | 1.00 | | |
| | CG/GG | 6 | 28 | 4.08 | 0.006 | 1.499-11.113 |
| Age, BMI & | CC | 48 | 65 | 1.00 | | |
| diabetes status | | | | | | |
| | CG/GG | 6 | 28 | 4.07 | 0.007 | 1.465-11.290 |

*All African-American females were CC (Pro/Pro) genotype; therefore, no tests of associations were done
**Referent group

TABLE 3B
Logistic regression analysis of PPAR gamma (P12A) recessive models with
PCAD/CAD in WHC Caucasian males

| | PPAR γ | | | | | |
|---|---|---|---|---|---|---|
| | (P12A) | N | | PPAR γ (P12A) Factor | | |
| Covariates | Genotype | (controls) | N (CAD) | OR | p-value | 95% C.I. |
| Age | CC | 52 | 199 | 1.00** | | |
| | CG/GG | 19 | 54 | 1.107 | 0.935 | 0.095-12.868 |
| Age and BMI | CC | 52 | 199 | 1.00 | | |
| | CG/GG | 19 | 54 | 0.746 | 0.344 | 0.407-1.367 |
| Age, BMI & | CC | 52 | 199 | 1.00 | | |
| diabetes status | | | | | | |
| | CG/GG | 19 | 54 | 0.752 | 0.360 | 0.409-1.383 |

**referent group

TABLE 3C
Logistic regression analysis of PPAR gamma (P12A) recessive models with MI in
WHC Caucasian* females

| | PPAR γ | | | | | |
|---|---|---|---|---|---|---|
| | (P12A) | | | PPAR γ (P12A) Factor | | |
| Covariates | Genotype | N (controls) | N (MI) | OR | p-value | 95% C.I. |
| Age | CC | 88 | 25 | 1.00** | | |
| | CG/GG | 19 | 15 | 2.519 | 0.017 | 1.182-5.369 |
| Age and BMI | CC | 88 | 25 | 1.00 | | |
| | CG/GG | 19 | 15 | 3.105 | 0.008 | 1.346-7.166 |
| Age, DMI & diabetes | CC | 88 | 25 | 1.00 | | |
| status | | | | | | |
| | CG/GG | 19 | 15 | 3.154 | 0.008 | 1.354-7.348 |

*All African-American females were CC (Pro/Pro) genotype, therefore no tests of associations were done
**referent group

TABLE 3D
Logistic regression analysis of PPAR gamma (P12A) recessive models with MI in
WHC Caucasian males

| | PPAR γ | | | | | |
|---|---|---|---|---|---|---|
| | (P12A) | N | | PPAR γ (P12A) Factor | | |
| Covariates | Genotype | (controls) | N (MI) | OR | p-value | 95% C.I. |
| Age | CC | 132 | 117 | 1.00** | | |
| | CG/GG | 44 | 29 | 0.871 | 0.586 | 0.531-1.430 |
| Age and BMI | CC | 132 | 117 | 1.00 | | |
| | CG/GG | 44 | 29 | 0.729 | 0.245 | 0.429-1.242 |
| Age, BMI & diabetes | CC | 132 | 117 | 1.00 | | |
| status | | | | | | |
| | CG/GG | 44 | 29 | 0.752 | 0.299 | 0.439-1.288 |

**referent group

TABLE 4A
Logistic regression analysis of PPAR gamma (P12A) recessive models with CAD
in SHS females*

| | PPAR γ | | | PPAR γ (P12A) Factor | | |
|---|---|---|---|---|---|---|
| | (P12A) | N | | | p- | |
| Covariates | Genotype | (controls) | N (CAD) | OR | value | 95% C.I. |
| Age and center | CC | 1587 | 168 | 1.00** | | |
| | CG/GG | 456 | 60 | 1.246 | 0.175 | 0.908-1.700 |
| Age, center and BMI | CC | 1583 | 168 | 1.00 | | |
| | CG/GG | 456 | 60 | 1.236 | 0.185 | 0.903-1.692 |
| Age, center, BMI and | CC | 1104 | 73 | 1.00 | | |
| fasting glucose | | | | | | |
| | CG/GG | 318 | 28 | 1.414 | 0.141 | 0.890-2.245 |

*includes only subjects without prevalent cardiovascular disease (CHD or stroke) at baseline
**referent group

TABLE 4B
Logistic regression analysis of PPAR gamma (P12A) recessive models with CAD in
SHS males*

| | PPAR γ | | | PPAR γ (P12A) Factor | | |
|---|---|---|---|---|---|---|
| | (P12A) | N | | | p- | |
| Covariates | Genotype | (controls) | N (CAD) | OR | value | 95% C.I. |
| Age and center | CC | 975 | 171 | 1.00** | | |
| | CG/GG | 269 | 37 | 0.793 | 0.236 | 0.540-1.164 |
| Age, center and BMI | CC | 967 | 171 | 1.00 | | |
| | CG/GG | 268 | 37 | 0.764 | 0.171 | 0.519-1.123 |
| Age, center, BMI and | CC | 735 | 108 | 1.00 | | |
| fasting glucose | | | | | | |
| | CG/GG | 194 | 18 | 0.623 | 0.083 | 0.365-1.063 |

*includes only subjects without prevalent cardiovascular disease (CHD or stroke) at baseline
**referent group

TABLE 4C
Logistic regression analysis of PPAR gamma (P12A) recessive models with MI in
SHS females*

| | PPAR γ | | | PPAR γ (P12A) Factor | | |
|---|---|---|---|---|---|---|
| | (P12A) | N | N | | p- | |
| Covariates | Genotype | (controls) | (MI) | OR | value | 95% C.I. |
| Age and center | CC | 1667 | 88 | 1.00** | | |
| | CG/GG | 482 | 34 | 1.373 | 0.130 | 0.910-2.072 |
| Age, center and BMI | CC | 1663 | 88 | 1.00 | | |
| | CG/GG | 482 | 34 | 1.366 | 0.137 | 0.904-2.062 |
| Age, center, BMI and | CC | 1132 | 45 | 1.00 | | |
| fasting glucose | | | | | | |
| | CG/GG | 325 | 21 | 1.825 | 0.030 | 1.059-3.146 |

*includes only subjects without prevalent cardiovascular disease (CHD or stroke) at baseline
**referent group

TABLE 4D
Logistic regression analysis of PPAR gamma (P12A) recessive models with MI in
SHS males*

| | PPAR γ | | | | | |
|---|---|---|---|---|---|---|
| | (P12A) | N | | PPAR γ (P12A) Factor | | |
| Covariates | Genotype | (controls) | N (MI) | OR | p-value | 95% C.I. |
| Age and center | CC | 1035 | 111 | 1.00** | | |
| | CG/GG | 284 | 22 | 0.737 | 0.212 | 0.456-1.190 |
| Age, center and BMI | CC | 1027 | 111 | 1.00 | | |
| | CG/GG | 283 | 22 | 0.703 | 0.151 | 0.435-1.138 |
| Age, center, BMI and | CC | 768 | 75 | 1.00 | | |
| fasting glucose | | | | | | |
| | CG/GG | 200 | 12 | 0.590 | 0.107 | 0.311-1.121 |

*includes only subjects without prevalent cardiovascular disease (CHD or stroke) at baseline
**referent group

### REFERENCES

1. Ross, R. Atherosclerosis—an inflammatory disease. N. Engl. J Med. 1999; 340:115-126.
2. Berg, A. H. and Scherer, P. E. Adipose tissue, inflammation, and cardiovascular disease. Circ Res. 2005; 96:939-949.
3. Auboeuf, D., Rieusset, J., Fajas, L., Vallier, P., Frering, V., Riou, J. P., Staels, B., Auwerx, J., Laville, M. Vidal, H. Tissue distribution and quantification of the expression of mRNAs of peroxisome proliferator-activated receptors and liver X receptor-alpha in humans: no alteration in adipose tissue of obese and NIDDM patients. Diabetes. 1997; 46:1319-1327.
4. Temelkova-Kurlctschiev, T., Hanefeld, M., Chinetti, G., Zawadzki, C., Haulon, S., Kubaszek, A., Koehler, C., Leonhardt, W., Staels, B. Laakso, M. Ala12Ala genotype of the peroxisome proliferator-activated receptor gamma2 protects against atherosclerosis. J. Clin. Endocrinol. Metab. 2004; 89:4238-4242.
5. Tontonoz, P., Hu, E., Devine, J., Beale, E. G. Spiegelman, B. M. PPAR gamma 2 regulates adipose expression of the phosphoenolpyruvate carboxykinase gene. Mol. Cell Biol. 1995; 15:351-357.
6. Barak, Y., Nelson, M. C., Ong, E. S., Jones, Y. Z., Ruiz-Lozano, P., Chien, K. R., Koder, A. Evans, R. M. PPAR gamma is required for placental, cardiac, and adipose tissue development. Mol. Cell. 1999; 4:585-595.
7. Chen, Z., Ishibashi, S., Perrey, S., Osuga, Ji, Gotoda, T., Kitamine, T., Tamura, Y., Okazaki, H., Yahagi, N., Iizuka, Y., Shionoiri, F., Ohashi, K., Harada, K., Shimano, H., Nagai, R. Yamada, N. Troglitazone inhibits atherosclerosis in apolipoprotein E-knockout mice: pleiotropic effects on CD36 expression and HDL. Arterioscler. Thromb. Vasc. Biol. 2001; 21:372-377.
8. Dormandy, J. A., Charbonnel, B., Eckland, D. J., Erdmann, E., Massi-Benedetti, M., Moules, I. K., Skene, A. M., Tan, M. H., Lefebvre, P. J., Murray, G. D., Standl, E., Wilcox, R. G., Wilhelmsen, L., Betteridge, J., Birkeland, K., Golay, A., Heine, R. J., Koranyi, L., Laakso, M., Mokan, M., Norkus, A., Pirags, V., Podar, T., Scheen, A., Scherbaum, W., Schernthaner, G., Schmitz, O., Skrha, J., Smith, U. Taton, J. Secondary prevention of macrovascular events in patients with type 2 diabetes in the PROactive Study (PROspective pioglitAzone Clinical Trial In macroVascular Events): a randomised controlled trial. Lancet. 2005; 366:1279-1289.
9. Deeb, S. S., Fajas, L., Nemoto, M., Pihlajamaki, J., Mykkanen, L., Kuusisto, J., Laakso, M., Fujimoto, W. Auwerx, J. A Pro12Ala substitution in PPARgamma2 associated with decreased receptor activity, lower body mass index and improved insulin sensitivity. Nat. Genet. 1998; 20:284-287.
10. Yen, C. J., Beamer, B. A., Negri, C., Silver, K., Brown, K. A., Yarnall, D. P., Burns, D. K., Roth, J. Shuldiner, A. R. Molecular scanning of the human peroxisome proliferator activated receptor gamma (hPPAR gamma) gene in diabetic Caucasians: identification of a Pro12Ala PPAR gamma 2 missense mutation. Biochem. Biophys. Res. Commun. 1997; 241:270-274.
11. Lohmueller, K. E., Pearce, C. L., Pike, M., Lander, E. S. Hirschhorn, J. N. Meta-analysis of genetic association studies supports a contribution of common variants to susceptibility to common disease. Nat. Genet. 2003; 33:177-182.
12. Al-Shali, K. Z., House, A. A., Hanley, A. J., Khan, H. M., Harris, S. B., Zinman, B., Mamakeesick, M., Fenster, A., Spence, J. D. Hegele, R. A. Genetic variation in PPARG encoding peroxisome proliferator-activated receptor gamma associated with carotid atherosclerosis. Stroke. 2004; 35:2036-2040.
13. Pischon, T., Pai, J. K., Manson, J. E., Hu, F. B., Rexrode, K. M., Hunter, D. Rimm, E. B. Peroxisome proliferator-activated receptor-gamma2 P12A polymorphism and risk of coronary heart disease in US men and women Arterioscler. Thromb. Vasc. Biol. 2005; 25:1654-1658.
14. Ridker, P. M., Cook, N. R., Cheng, S., Erlich, H. A., Lindpaintner, K., Plutzky, J. Zee, R. Y. Alanine for proline substitution in the peroxisome proliferator-activated receptor gamma-2 (PPARG2) gene and the risk of incident myocardial infarction. Arterioscler. Thromb. Vasc. Biol. 2003; 23:859-863.
15. Altshuler, D., Hirschhorn, J. N., Klannemark, M., Lindgren, C. M., Vohl, M. C., Nemesh, J., Lane, C. R., Schaffner, S. F., Bolk, S., Brewer, C., Tuomi, T., Gaudet, D., Hudson, T. J., Daly, M., Groop, L. Lander, E. S. The common PPARgamma Pro12Ala polymorphism is associated with decreased risk of type 2 diabetes. Nat. Genet. 2000; 26:76-80.
16. L ee, E. T., Cowan, L. D., Welty, T. K., Sievers, M., Howard, W. J., Oopik, A., Wang, W., Yeh, J., Devereux, R. B., Rhoades, E. R., Fabsitz, R. R., Go, O. Howard, B. V. All-cause mortality and cardiovascular disease mortality in three American Indian populations, aged 45-74 years, 1984-1988. The Strong Heart Study. Am. J Epidemiol. 1998; 147:995-1008.
17. Howard, B. V., Welty, T. K., Fabsitz, R. R., Cowan, L. D., Oopik, A. J., Le, N. A., Yeh, J., Savage, P. J. Lee, E. T. Risk factors for coronary heart disease in diabetic and nondiabetic Native Americans. The Strong Heart Study. Diabetes. 1992; 41 Suppl 2:4-11.
18. Bishop-Bailey, D. Peroxisome proliferator-activated receptors in the cardiovascular system. Br. J. Pharmacol. 2000; 129:823-834.
19. Tontonoz, P., Hu, E. Spiegelman, B. M. Regulation of adipocyte gene expression and differentiation by peroxisome proliferator activated receptor gamma. Curr. Opin. Genet. Dev. 1995; 5:571-576.
20. Plutzky, J. Peroxisome proliferator-activated receptors in vascular biology and atherosclerosis: emerging insights for evolving paradigms. Curr. Atheroscler. Rep. 2000; 2:327-335.
21. Tontonoz, P. and Nagy, L. Regulation of macrophage gene expression by peroxisome-proliferator-activated receptor gamma: implications for cardiovascular disease. Curr. Opin. Lipidol. 1999; 10:485-490.
22. Claudel, T., Leibowitz, M. D., Fievet, C., Tailleux, A., Wagner, B., Repa, J. J., Torpier, G., Lobaccaro, J. M., Paterniti, J. R., Mangelsdorf, D. J., Heyman, R. A. Auwerx, J. Reduction of atherosclerosis in apolipoprotein E knockout mice by activation of the retinoid X receptor. Proc. Natl. Acad. Sci. U.S.A. 2001; 98:2610-2615.
23. Corti, R., Osende, J. I., Fallon, J. T., Fuster, V., Mizsei, G., Jneid, H., Wright, S. D., Chaplin, W. F. Badimon, J. J. The selective peroxisomal proliferator-activated receptor-gamma agonist has an additive effect on plaque regression in combination with simvastatin in experimental atherosclerosis: in vivo study by high-resolution magnetic resonance imaging. J. Am. Coll. Cardiol. 2004; 43:464-473.
24. Tanaka, T., Fukunaga, Y., Itoh, H., Doi, K., Yamashita, J., Chun, T. H., Inoue, M., Masatsugu, K., Saito, T., Sawada, N., Sakaguchi, S., Arai, H. Nakao, K. Therapeutic potential of thiazolidinediones in activation of peroxisome proliferator-activated receptor gamma for monocyte recruitment and endothelial regeneration. Eur. J. Pharmacol. 2005; 508:255-265.
25. Marx, N., Froehlich, J., Siam, L., Ittner, J., Wierse, G., Schmidt, A., Scharnagl, H., Hombach, V. Koenig, W. Antidiabetic PPAR gamma-activator rosiglitazone reduces MMP-9 serum levels in type 2 diabetic patients with coronary artery disease. Arterioscler. Thromb. Vasc. Biol. 2003; 23:283-288.
26. Wang, G., Wei, J., Guan, Y., Jin, N., Mao, J. Wang, X. Peroxisome proliferator-activated receptor-gamma agonist rosiglitazone reduces clinical inflammatory responses in type 2 diabetes with coronary artery disease after coronary angioplasty. Metabolism. 2005; 54:590-597.
27. Masugi, J., Tamori, Y., Mori, H., Koike, T. Kasuga, M. Inhibitory effect of a proline-to-alanine substitution at codon 12 of peroxisome proliferator-activated receptor-gamma 2 on thiazolidinedione-induced adipogenesis. Biochem. Biophys. Res. Commun. 2000; 268:178-182.
28. Dugi, K. A., Brandauer, K., Schmidt, N., Nau, B., Schneider, J. G., Mentz, S., Keiper, T., Schaefer, J. R., Meissner, C., Kather, H., Bahner, M. L., Fiehn, W. Kreuzer, J. Low hepatic lipase activity is a novel risk factor for coronary artery disease. Circulation. 2001; 104:3057-3062.
29. Benlian, P., De Gennes, J. L., Foubert, L., Zhang, H., Gagne, S. E. Hayden, M. Premature atherosclerosis in patients with familial chylomicronemia caused by mutations in the lipoprotein lipase gene. N. Engl. J. Med. 1996; 335:848-854.
30. Clee, S. M., Bissada, N., Miao, F., Miao, L., Marais, A. D., Henderson, H. E., Steures, P., McManus, J., McManus, B., LeBoeuf, R. C., Kastelein, J. J. Hayden, M. R. Plasma and vessel wall lipoprotein lipase have different roles in atherosclerosis. J. Lipid Res. 2000; 41:521-531.
31. Wittrup, H. H., Tybjaerg-Hansen, A., Steffensen, R., Deeb, S. S., Brunzell, J. D., Jensen, G. Nordestgaard, B. G. Mutations in the lipoprotein lipase gene associated with ischemic heart disease in men. The Copenhagen city heart study. Arterioscler. Thromb. Vasc. Biol. 1999; 19:1535-1540.
32. Weiss, E. P., Kulaputana, O., Ghiu, I. A., Brandauer, J., Wohn, C. R., Phares, D. A., Shuldiner, A. R. Hagberg, J. M. Endurance training-induced changes in the insulin response to oral glucose are associated with the peroxisome proliferator-activated receptor-gamma2 Pro12Ala genotype in men but not in women. Metabolism. 2005; 54:97-102.
33. Fillingim, R. B., Kaplan, L., Staud, R., Ness, T. J., Glover, T. L., Campbell, C. M., Mogil, J. S. Wallace, M. R. The A118G single nucleotide polymorphism of the mu-opioid receptor gene (OPRM1) is associated with pressure pain sensitivity in humans. J. Pain. 2005; 6:159-167.
34. Thomson, P. A., Wray, N. R., Thomson, A. M., Dunbar, D. R., Grassie, M. A., Condie, A., Walker, M. T., Smith, D. J., Pulford, D. J., Muir, W., Blackwood, D. H. Porteous, D. J. Sex-specific association between bipolar affective disorder in women and GPR50, an X-linked orphan G protein-coupled receptor. Mol. Psychiatry. 2005; 10:470-478.
35. Ellis, J. A., Infantino, T. Harrap, S. B. Sex-dependent association of blood pressure with oestrogen receptor genes ERalpha and ERbeta. J. Hypertens. 2004; 22:1127-1131.
36. L i, A. C., Brown, K. K., Silvestre, M. J., Willson, T. M., Palinski, W. Glass, C. K. Peroxisome proliferator-activated receptor gamma ligands inhibit development of atherosclerosis in LDL receptor-deficient mice. J. Clin. Invest. 2000; 106:523-531.

What is claimed is:

1. A method of assessing or aiding in assessing the risk that a woman will develop CAD, comprising obtaining a biological sample from a woman to be assessed for the risk of developing CAD and analyzing the sample for the presence of at least one copy of the Ala allele of the human PPARγ P12 gene, wherein if the sample contains at least one copy of the Ala allele, the woman is at increased risk for developing CAD.

2. The method of claim 1, wherein the CAD is premature CAD.

3. The method of claim 1 or 2, wherein the biological sample is blood, plasma or serum.

4. A method of predicting or aiding in predicting the likelihood that a woman will develop CAD, comprising obtaining a biological sample from a woman for whom the risk of developing CAD is to be predicted and determining if at least one copy of the Ala allele of the human PPARγ P12 gene is present in the sample, wherein if the sample contains at least one copy of the Ala allele, the likelihood that the woman will develop CAD is greater than if the sample does not contain the Ala allele.

5. The method of claim 4, wherein the CAD is premature CAD.

6. The method of claim 4 or 5, wherein the sample is blood, serum or plasma.

7. A method of assessing or aiding in assessing the risk that a woman will develop CAD, comprising obtaining a biological sample from a woman to be assessed for the risk of developing CAD and analyzing the sample for the presence of at least one copy of the Ala allele of the human PPARγ P12 gene, wherein if the sample contains at least one copy of the Ala allele, the woman is at increased risk for developing CAD.

8. The method of claim 7, wherein the CAD is premature CAD.

9. The method of claim 7 or 8, wherein the woman is a Caucasian woman.

10. The method of claim 7, 8 or 9, wherein the biological sample is blood, plasma or serum.

11. A method of predicting or aiding in predicting the likelihood that a woman will develop CAD, comprising obtaining a biological sample from a woman for whom the risk of developing CAD is to be predicted and determining if at least one copy of the Ala allele of the human PPARγ P12 gene is present in the sample, wherein if the sample contains at least one copy of the Ala allele, the likelihood that the woman will develop CAD is greater than if the sample does not contain the Ala allele.

12. The method of claim 11, wherein the CAD is premature CAD.

13. The method of claim 11 or 12, wherein the woman is a Caucasian woman.

14. The method of claim 11, 12 or 13, wherein the sample is blood, serum or plasma.

15. A method of assessing or aiding in assessing the risk that a man will develop premature CAD, comprising obtaining a biological sample from a man to be assessed for the risk of developing premature CAD and analyzing the sample for the presence of at least one copy of the G allele of the human resistin gene, wherein if the sample contains at least one copy of the G allele, the man is at increased risk for developing premature CAD.

16. The method of claim 15, wherein the biological sample is blood, plasma or serum.

17. A method of predicting or aiding in predicting the likelihood that a man will develop premature CAD, comprising obtaining a biological sample from a man for whom the risk of developing premature CAD is to be predicted and determining if at least one copy of the G allele of the human resistin gene is present in the sample, wherein if the sample contains at least one copy of the G allele, the likelihood that the man will develop premature CAD is greater than if the sample does not contain the G allele.

18. The method of claim 17, wherein the sample is blood, serum or plasma.

19. A method of assessing or aiding in assessing the risk that a man will develop premature CAD, comprising obtaining a biological sample from a man to be assessed for the risk of developing premature CAD and analyzing the sample for the presence of at least one copy of the G allele of the human resistin gene, wherein if the sample contains at least one copy of the G allele, the man is at increased risk for developing premature CAD.

20. The method of claim 19, wherein the biological sample is blood, plasma or serum.

21. A method of predicting or aiding in predicting the likelihood that a man will develop premature CAD, comprising obtaining a biological sample from a man for whom the risk of developing premature CAD is to be predicted and determining if at least one copy of the G allele of the human resistin gene is present in the sample, wherein if the sample contains at least one copy of the G allele, the likelihood that the man will develop premature CAD is greater than if the sample does not contain the G allele.

22. The method of claim 21, wherein the sample is blood, serum or plasma.

23. A method of assessing or aiding in assessing the risk that a woman will experience MI, comprising obtaining a biological sample from a woman to be assessed for the risk of experiencing MI and analyzing the sample for the presence of at least one copy of the Ala allele of the human PPARγ P12 gene, wherein if the sample contains at least one copy of the Ala allele, the woman is at increased risk for experiencing MI.

24. The method of claim 23, wherein the woman is a caucasian woman or an African American woman.

25. The method of claim 23, wherein the biological sample is blood, plasma or serum.

26. A method of predicting or aiding in predicting the likelihood that a woman will experience MI, comprising obtaining a biological sample from a woman for whom the risk of experience MI is to be predicted and determining if at least one copy of the Ala allele of the human PPARγ P12 gene is present in the sample, wherein if the sample contains at least one copy of the Ala allele, the likelihood that the woman will experience MI is greater than if the sample does not contain the Ala allele.
